(19) United States
(12) Patent Application Publication
(10) Pub. No.: US 2020/0104715 A1
(43) Pub. Date: Apr. 2, 2020

(54) TRAINING OF NEURAL NETWORKS BY INCLUDING IMPLEMENTATION COST AS AN OBJECTIVE

(71) Applicant: Xilinx, Inc., San Jose, CA (US)

(72) Inventors: Kristof Denolf, Longmont, CO (US); Nicholas Fraser, Dublin (IE); Kornelis A. Vissers, Sunnyvale, CA (US); Giulio Gambardella, Portmarnock (IE)

(73) Assignee: Xilinx, Inc., San Jose, CA (US)

(21) Appl. No.: 16/147,478

(22) Filed: Sep. 28, 2018

(51) Int. Cl. G06N 3/08; G06N 3/04

(52) U.S. Cl. CPC G06N 3/082; G06N 3/0445

(57) ABSTRACT

An example method of implementing a neural network includes selecting a first neural network architecture from a search space and training the neural network having the first neural network architecture to obtain an accuracy and an implementation cost. The implementation cost is based on a programmable device of an inference platform. The method further includes selecting a second neural network architecture from the search space based on the accuracy and the implementation cost, and outputting weights and hyperparameters for the neural network having the second neural network architecture.

### TECHNICAL FIELD

Examples of the present disclosure generally relate to neural networks and, in particular, to training of neural network by including implementation cost as an objective.

### BACKGROUND

Machine learning is the science of inducing computing systems to act without being explicitly programmed. Classical machine learning includes various clustering and classification techniques, including K-means clustering, linear and logistic regressions, stochastic gradient decent, association rule learning, and the like. Deep learning is a newer frontier in machine learning. Deep learning is a class of machine learning algorithms that uses multiple layers of nonlinear processing units for feature extraction and transformation. Deep learning algorithms can be unsupervised (e.g., pattern analysis) or supervised (e.g., classification). The deep learning algorithm can be implemented using layers of an artificial neural network (ANN) (referred to herein as a “neural network”).

In general, a neural network is a collection of nodes (i.e., the “neurons”) that are connected in a graph. A node in a neural network computes a sum of weighted inputs and adds an optional bias to the sum. The output of the node is a function of the final sum (referred to as an “activation function”), Example activation functions include the sigmoid function, the hyperbolic tangent (tank) function, the Rectified Linear Unit (ReLU) function, and the identity function. Neural network models are often organized into layers of nodes, which define a specific topology, and corresponding weights and biases. The weights and biases are referred to as network parameters.

In general, a neural network includes an input layer and an output layer and can optionally include one or more hidden layers between the input and output layers. A neural network used in deep learning applications typically includes many hidden layers, which gives rise to the term deep neural network (DNN). The layers of a neural network can be densely connected (e.g., each node in a layer is fully connected to all nodes in a previous layer) or sparsely connected (e.g., each node in a layer is connected to only a portion of the nodes in a previous layer). A convolutional neural network (CNN) is a type of DNN that includes one or more sparsely connected layers, referred to as convolutional layers. A CNN is well-suited for processing image or video data. Other types of DNNs include recurrent neural network (RNNs), which are well-suited for processing speech and text data.

Neural networks of any topology or type need the correct values of the network parameters across all layers in order to adapt the network to a specific task. A supervised training procedure can be used to determine a set of network parameters that yields desired accuracy for the specified task. Training involves running a training data set through a forward path of the network (forward propagation) and updating the weights through a backward path of the network (backward propagation) to compensate for prediction errors. The trained neural network is then deployed to perform the specified task on input data sets (referred to as inference). The computing platform used to train a neural network (training platform) is often more highly performant than the computing platform used for inference (inference platform). The inference platform, however, is often more power efficient than the training platform. Conventional training techniques do not account for architectural aspects of the inference platform, which can result in less than optimal implementations of the neural network for the target inference platform.

### SUMMARY

Techniques for training of neural network by including implementation cost as an objective are described. In an example, a method of implementing a neural network includes: selecting a first neural network architecture from a search space; training the neural network having the first neural network architecture to obtain an accuracy and an implementation cost, the implementation cost based on a programmable device of an inference platform; selecting a second neural network architecture from the search space based on the accuracy and the implementation cost; and outputting weights and hyperparameters for the neural network having the second neural network architecture.

In another example, a non-transitory computer readable medium comprising instructions, which when executed in a computer system, causes the computer system to carry out a method of implementing a neural network includes: selecting a first neural network architecture from a search space; training the neural network having the first neural network architecture to obtain an accuracy and an implementation cost, the implementation cost based on a programmable device of an inference platform; selecting a second neural network architecture from the search space based on the accuracy and the implementation cost; and outputting weights and hyperparameters for the neural network having the second neural network architecture.

In another example, a computer system includes: a memory having program code stored therein; and a processor, configured to execute the program code, to implement a neural network by: selecting a first neural network architecture from a search space; training the neural network having the first neural network architecture to obtain an accuracy and an implementation cost, the implementation cost based on a programmable device of an inference platform; selecting a second neural network architecture from the search space based on the accuracy and the implementation cost; and outputting weights and hyperparameters for the neural network having the second neural network architecture.

These and other aspects may be understood with reference to the following detailed description.

### BRIEF DESCRIPTION OF THE DRAWINGS

So that the manner in which the above recited features can be understood in detail, a more particular description, briefly summarized above, may be had by reference to example implementations, some of which are illustrated in the appended drawings. It is to be noted, however, that the appended drawings illustrate only typical example implementations and are therefore not to be considered limiting of its scope.

FIG. 1 is a block diagram depicting a system for training and implementing a neural network according to an example.

FIG. 2 is a block diagram depicting a computing system according to an example.

FIG. 3 is a method of training a neural network according to an example.

FIG. 4 is a method of training a neural network according to another example.

FIG. 5 is a method of training a neural network according to another example.

FIG. 6 is a flow diagram depicting a method of implementing an inference platform according to an example.

FIG. 7 is a block diagram depicting a programmable integrated circuit (IC) according to an example.

FIG. 8 is a block diagram depicting a System-on-Chip (SoC) implementation of the programmable IC of FIG. 7

FIG. 9 illustrates a field programmable gate array (FPGA) implementation of the programmable IC of FIG. 7.

To facilitate understanding, identical reference numerals have been used, where possible, to designate identical elements that are common to the figures. It is contemplated that elements of one example may be beneficially incorporated in other examples.

### DETAILED DESCRIPTION

Various features are described hereinafter with reference to the figures. It should be noted that the figures may or may not be drawn to scale and that the elements of similar structures or functions are represented by like reference numerals throughout the figures. It should be noted that the figures are only intended to facilitate the description of the features. They are not intended as an exhaustive description of the claimed invention or as a limitation on the scope of the claimed invention. In addition, an illustrated example need not have all the aspects or advantages shown. An aspect or an advantage described in conjunction with a particular example is not necessarily limited to that example and can be practiced in any other examples even if not so illustrated or if not so explicitly described.

Techniques for training of neural network by including implementation cost as an objective are described. The techniques provide a cost-aware architectural search of a neural network topology. As such, the training of a neural network no longer only targets maximizing the accuracy of the neural network at a certain task. Rather, the neural network training balances accuracy against the implementation cost of the neural network, which is included as another objective in the training. In this manner, the training becomes a multi-objective search, where not only the values of the weights are trained, but also the topology and certain implementation-related attributes of the neural network are found.

The techniques described herein address the high compute/memory demands in neural networks and its actual implementation into a hardware backend during the training phase. The techniques include deriving/alternating the network topology, its hyperparameters, and certain implementation related attributes by making the (inference) implementation cost of the neural network an extra objective during training (next to the initial, often accuracy related, objectives), as well as other properties such as error tolerance (e.g., in case of safety-critical applications). Conventional training does not account for architectural aspects of the inference platform. Complexity optimization techniques focus on reducing memory bandwidth by pruning/compressing weights and/or feature maps and reducing the precision (bit width) of the weight and/or feature maps. Reinforcement learning provides for multi-objective optimization, but without adding the implementation cost of the neural network itself as an objective. The techniques described herein for training using implementation cost as an objective are complementary to those techniques. These and further aspects of optimizing network parameters and/or feature maps based on architecture constraints of the inference platform are described below with respect to the drawings.

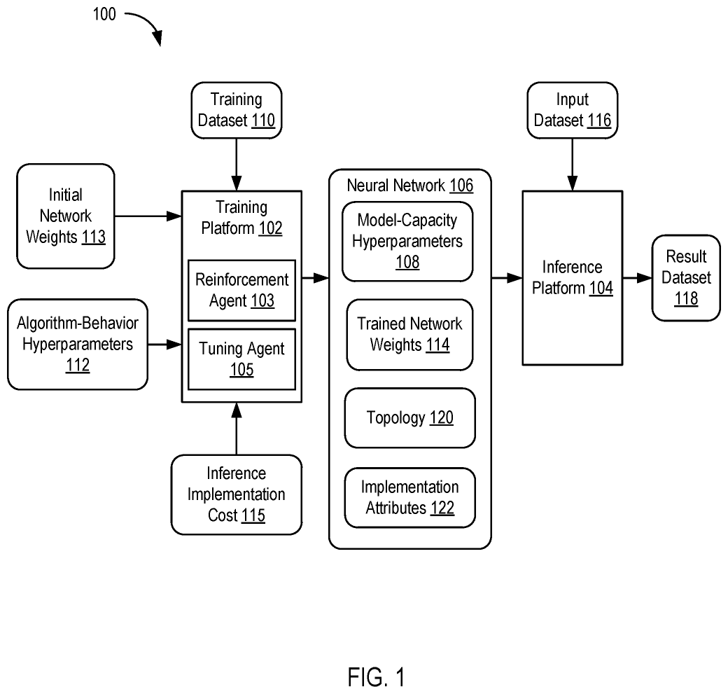

FIG. 1 is a block diagram depicting a system 100 for training and implementing a neural network according to an example. The system 100 includes a training platform 102 and an inference platform 104. The training platform 102 comprises hardware and software configured to train a neural network 106 for a specified task (e.g., image classification, object detection, etc.). As described below, the training platform includes a reinforcement agent 103 and a tuning agent 105. The inference platform 104 includes hardware and/or software configured to implement the neural network 106 to perform the specified task. Examples of the training platform 102 and the inference platform 104 are described below.

The implementation efficiency of a neural network implementation can be measured by different costs, such as throughput, energy, size, error tolerance, and the like, or combinations thereof. This cost is the result of different design aspects, such as the number of operations, bandwidth, data locality, scheduling on the hardware backend, and the like. These aspects are related to the characteristics of the training algorithm, where a better algorithmic performance often leads to higher implementation costs (Pareto principle). Typically, maximizing the algorithmic accuracy for a specific task/capability is the main objective during training. Additionally, the network topology is often engineered, and training focuses on finding the correct values of all the weights in the different layers of the neural network. These weights are then used during inference to perform this task/capability. The configuration of the training algorithm is controlled by “algorithmic-behavior” hyperparameters. Additionally, the term hyperparameters is also used for parameters that define the capacity of the neural network (e.g., the number of hidden layers in a neural network) and hence are related to the network topology. These hyperparameters are referred to as “model-capacity” hyperparameters herein and include all implementation attributes (e.g., bit width).

The training platform 102 receives a training dataset 110 and initial network weights 113. The training dataset 110 includes data for training the neural network 106 to generate trained network weights 114. For example, if the neural network 106 is configured to classify images, the training dataset 110 can be a set of pre-classified images. The initial network weights 113 include initial values for the weights of the neural network 106. In an example, the training platform 102 also includes an input to receive algorithm-behavior hyperparameters 112. The algorithm-behavior hyperparameters 112 include learning rate, early stop criteria, and the like. The training platform 102 also includes an input to receive inference implementation cost 115. The training platform 102 uses the inference implementation cost 115 as a training objective to learn optimal weights 114, network topology 120, model-capacity hyperparameters 108, and implementation attributes 122 (e.g., weight or tensor element bit widths, number formats, and the like) achieving the best trade-off in the accuracy, implementation cost Pareto space.

A minimum accuracy can be enforced while exploring this Pareto space. In this case, the training looks for the lowest cost implementation that at least achieves the expected accuracy. The combined accuracy and inference-specific implementation cost training objective is applicable to any compute platform (e.g., CPUs, GPUs, ASSPs, FPGAs, ACAPs, etc. or any combination thereof). Inference-specific implementation costs include throughput, energy, size, error tolerance, and the like or a combination thereof. Such inference-specific implementation costs are also referred to herein more generally as implementation costs. The flexible architecture of FPGAs is ideally suited to enable this combined accuracy and implementation cost training objective, since all architectural design parameters/aspects (e.g., bit widths, number of processing elements, etc.) are unfixed and hence available to be learned during training.

The topology 120 generally includes an arrangement of neurons. For example, the topology 120 can include a plurality of layers of neurons. The layers generally include an input layer, an output layer, and zero or more hidden layers. Each neuron includes a plurality of inputs and an output. The plurality of inputs for each neuron are associated with a plurality of weights. Each neuron further includes a bias associated with its output. The weights and biases of the neural network 106 are referred to as trained network weights 114. For a given layer, the inputs of its neurons are referred to as input feature maps and the outputs of its neurons are referred to as output feature maps. Input feature maps and output feature maps are generally referred to as “feature maps.”

The inference platform 104 implements the neural network 106. An input dataset 116 includes the data to be processed by the neural network 106. For example, if the neural network is configured to classify images, the input dataset 116 can include images to be classified. The inference platform 104 generates a result dataset 118. For example, in an image classification scheme, the result dataset 118 includes classifications for images in the input dataset 116. Since the neural network 106 has been optimized based on implementation cost of the inference platform 104, the neural network 106 can be implemented efficiently by the inference platform 104, taking advantage of its features, elements, and limitations that were captured by the inference implementation cost 115.

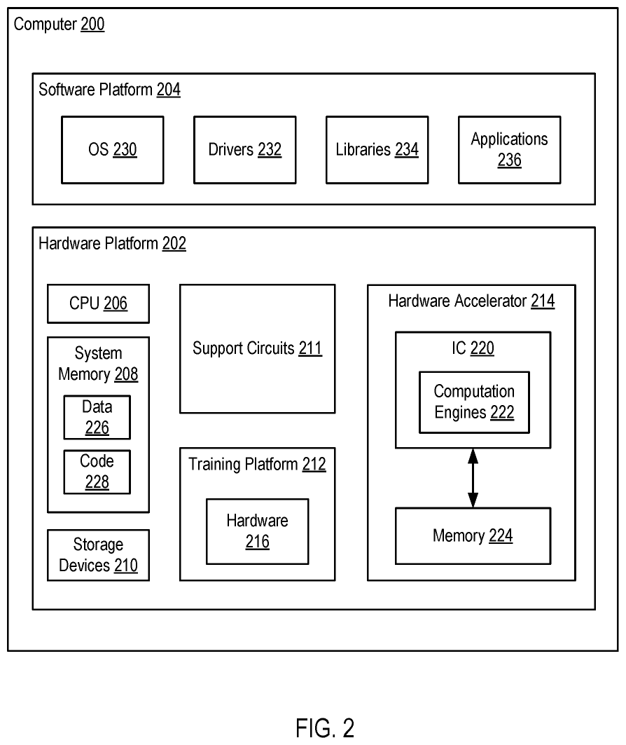

FIG. 2 is a block diagram depicting a computing system (“computer 200”) according to an example. The computer 200 includes a software platform 204 executing on a hardware platform 202. The hardware platform 202 includes a central processing unit (CPU) 206, a system memory 208, storage devices 210, support circuits 211, a training platform 212, and a hardware accelerator 214. The software platform 204 includes an operating system (OS) 230, drivers 232, libraries 234, and applications 236.

In an example, the CPU 206 can be any type of general-purpose central processing unit (CPU), such as an x86-based processor, ARM®-based processor, or the like. The CPU 206 can include one or more cores and associated circuitry (e.g., cache memories, memory management units (MMUs), interrupt controllers, etc.). The CPU 206 is configured to execute program code that perform one or more operations described herein and which can be stored in the system memory 208 and/or the storage devices 210. The support circuits 211 include various devices that cooperate with the CPU 206 to manage data flow between the CPU 206, the system memory 208, the storage devices 210, the training platform 212, the hardware accelerator 214, or any other peripheral device. For example, the support circuits 211 can include a chipset (e.g., a north bridge, south bridge, platform host controller, etc.), voltage regulators, firmware (e.g., a BIOS), and the like. In some examples, the CPU 206 can be a System-in-Package (SiP), System-on-Chip (SoC), or the like, which absorbs all or a substantial portion of the functionality of the chipset (e.g., north bridge, south bridge, etc.). In another example, the CPU 206 can be a vector processor or can include a vector processor.

The system memory 208 is a device allowing information, such as executable instructions and data, to be stored and retrieved. The system memory 208 can include, for example, one or more random access memory (RAM) modules, such as double-data rate (DDR) dynamic RAM (DRAM). The system memory 208 can store data 226 and program code (“code 228”) processed and executed by the CPU 206 to implement the software platform 204. The storage devices 210 includes local storage devices (e.g., one or more hard disks, flash memory modules, solid state disks, and optical disks) and/or a storage interface that enables the computer 200 to communicate with one or more network data storage systems. The hardware platform 202 can include various other conventional devices and peripherals of a computing system, such as graphics cards, universal serial bus (USB) interfaces, and the like.

The training platform 212 includes hardware 216, which can include processor(s), memory, input/output (IO) circuits, and the like. In an example, hardware 216 includes a graphics processing unit (GPU) and associated support circuitry. In another example, hardware 216 can include an application specific integrated circuit (ASIC), programmable IC, or the like along with associated support circuitry. In an example, training platform 212 is more performant than the hardware accelerator 214, but also consumes more energy than the hardware accelerator 214. The training platform 212 can be used to train neural networks.

The hardware accelerator 214 includes an IC 220 and memory 224. The IC 220 includes computation engines 222. In an example, the IC 220 is a programmable IC, such as a field programmable gate array (FGPA) or a system-on-chip (SoC) having an FPGA therein. The computation engines 222 can be programmed in the IC 220. In another example, the IC 220 is an ASIC or the like, where the computation engines 222 are dedicated circuitry therein. The hardware accelerator 214 can be used in an inference platform for neural networks.

The OS 230 can be any commodity operating system known in the art, such as such as Linux®, Microsoft Windows®, Mac OS®, or the like. The drivers 232 and libraries 234 comprise software that provide application programming interfaces (APIs) to the training platform 212 and the hardware accelerator 214 for command and control thereof. The applications 236 include software that trains neural networks on the training platform 212 and implements neural networks on the hardware accelerator 214. The applications 236 communicate with the training platform 212 and the hardware accelerator 214 through the drivers 232 and libraries 234.

Including the implementation cost as a goal in training makes the training a multi-objective problem. Techniques are described below for multi-objective optimization to combine the network accuracy and implementation cost. Three examples of training approaches for this implementation and accuracy driven neural network search are described: (1) using reinforcement learning; (2) using evolutionary based algorithms; and (3) using hyperparameter analysis/optimization. Techniques for reducing the size of the neural network architecture search space are also described.

### Multi-Objective Optimization

The inclusion of inference implementation cost when evaluating the performance of networks means there are at least two objectives that are to be optimized. As such, multiple objectives should be balanced in a meaningful way. For example, assume the accuracy of the network is given by classification error, C_{E}, and the estimated implementation cost is given by the time taken to process a new input, C_{T}. If minimizing C_{T} is given too much importance, then it is possible an optimizer will produce a network with zero layers, zero operations, and zero memory requirements. This could yield a network that has C_{T}=0, despite incurring a significantly high C_{E}. Multi-objective optimization aims to balance C_{E} and C_{T} to give a desirable solution.

A general formulation of multi-objective optimization is as follows:

$${min}_{x} ( {f}_{1} ( x ) , {f}_{2} ( x ) , … , {f}_{k} ( x ) ) s . t . x ∈ X ,$$

where f₁, . . . , fₓ are functions that define the cost of each objective that is being optimized, x is a vector representing the current solution, and X is the search space of all possible solutions. In the examples described herein, x represents a neural network topology and its associated hyperparameters (i.e., the model-capacity hyperparameters 108). The functions represent metrics of interest of the current neural network topology in relation to its accuracy and implementation/hardware cost. For accuracy, these functions include mean squares error (MSE), classification error, lₚ norm, hingle loss, or a similar metric suitable for the target domain. For implementation/hardware cost, these functions include memory requirements, bandwidth requirements, clock cycles, datapath width, quantization scheme, arithmetic style, number formats, silicon area, and energy consumption, and error tolerance.

In some cases, the objection functions cannot be easily combined mathematically in an understandable way. In these cases, when comparing two solutions x₁ and x₂, x₁ is a better solution than x₂ if fᵢ(x₁)<fᵢ(x₂)∀i. If no better solution can be found than x₁, then x₁ is considered to be a Pareto optimal solution. In other cases, multiple objective functions can be combined to form a single objective function that aims to encapsulate the tradeoffs of multiple objectives. This is known as scalarization and is formulated as follows in the general case:

$${min}_{x} ( g ( {f}_{1} ( x ) , {f}_{2} ( x ) , … , {f}_{k} ( x ) ) ) s . t . x ∈ X ,$$

where g∈R^{k}→R. Common examples of g include:
Linear scalarization, g=Σwᵢfᵢ(x), where wᵢ>0 is a weight associated with each objective function; and
Lₚ norm, g=∥f−z∥ₚ, where f={f₁(x), f₂(x), . . . , fₖ(x)}, and z∈R^{k} is a vector of ideal cost values.
   Depending on the optimizer of choice (e.g., described below), the object functions may need to be semi-differentiable, such as MSE, cross-entropy, and hinge loss. Three learning techniques for cost-aware architecture search are introduced below. Note that each of these techniques can be used in combination with each other.

The listed examples show implementation cost C as an additional optimization cost (next to accuracy R). This is a generic representation of the inference-specific implementation costs. It can represent a single implementation cost, like energy E or error tolerance T, etc. or any combination of costs.

### Reinforcement Learning Based Architecture Search

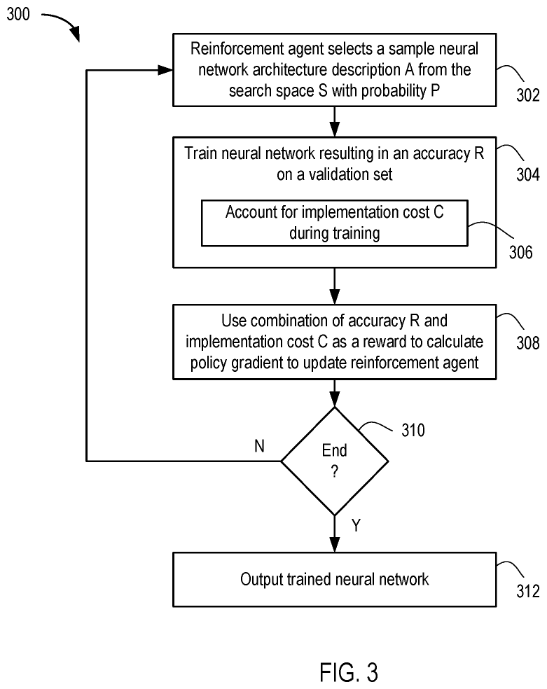

FIG. 3 is a method 300 of training a neural network according to an example. The method 300 begins at step 302, where a reinforcement agent 103 selects a sample neural network architecture description A from the search space S with probability P. The topology of a neural network (e.g., its structure and connectivity) can be described in a text format (e.g., prototxt or any other presentation used by neural network or machine learning frameworks). The neural network description is extended with implementation specific attributes (e.g., bit width of the tensor elements, number format, scheduling, etc.). The extended neural network description becomes the neural network architecture description.

At step 304, the training platform trains the neural network resulting in an accuracy R on a validation set. Since the neural network architecture description includes implementation attributes, the implementation cost C (based on the inference platform) can be measured or estimated/modeled (step 306). At step 308, the training platform uses a combination of accuracy R and implementation cost C as a reward to calculate a policy gradient to update the reinforcement agent 103. At step 310, the reinforcement agent 103 determines whether an end condition has been met for training. If not, the method 300 repeats, selecting another network architecture description from the search space S. It should be understood that the method 300, when selecting the next network architecture for processing, can select the same network architecture as a previous iteration. That is, the same network architecture can be used in multiple training iterations. Otherwise, the method 300 proceeds to step 312, where the training platform outputs the trained neural network.

In an example, the reinforcement agent 103 may be a machine learning algorithm tuned for sequence prediction, such as a recurrent neural network (RNN). This RNN takes as input the parameters of the previous network layer and produces a prediction for the parameters of the subsequent layer. The RNN continues in this fashion until a stopping criterion is reached. Example stopping criterion include: a certain number of layers is reached, or a certain hardware cost is reached (e.g., memory usage/number of operations). If a semi-differentiable objection function is chosen for network accuracy and implementation cost, some parameters may be updated by differentiating them with respect to the objective function. For other parameters, a policy is defined for gradients.

### Evolution Based Architecture Search

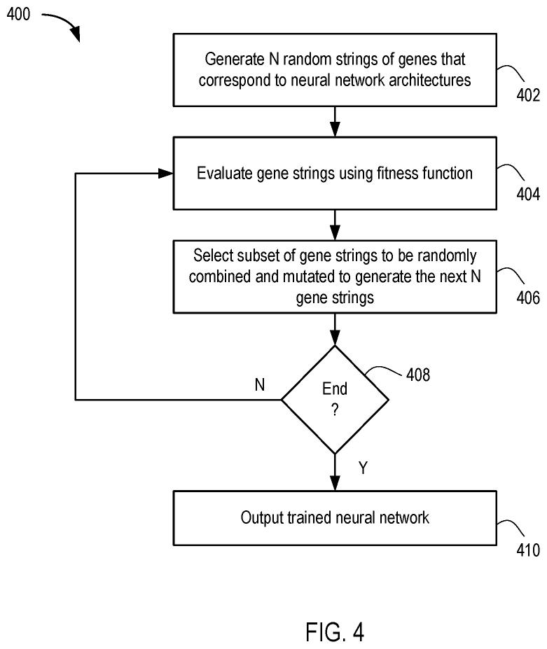

FIG. 4 is a block diagram depicting a method 400 of training a neural network according to another example. The method 400 may be implemented by the training platform. An alternative approach to an architecture search is to use an evolutionary based algorithm. In order to use evolutionary algorithms to perform the architecture search, two things are required: 1) an encoding of a neural network architecture into genes; and 2) a fitness function to evaluate the performance of a particular structure. The fitness function can be any function described above in the multi-objective optimization section, including scalarized or multi-objective functions. The evolutionary algorithm understands the implementation cost of such networks. In this case, the evolutionary algorithm can be used to find an optimal solution (scalarized) or a series of pareto optimal solutions, or close approximations. To encode a neural network architecture into genes, neural network descriptions can transformed into an alphabet. This can be an equivalent mapping to network design protocols, such as caffe's prototxt, written in a compact way to make an algorithm more conducive to evolutionary algorithms. Neural network layers, graph connections, and individual neurons and synapses can all be expressed as genes.

The basic methodology of evolutionary algorithms is to generate N random strings of genes (which correspond to neural network architectures) (step 402). These architectures are then evaluated using a fitness function, which may require training each network architecture individually (step 404). At this point, a subset of the architectures are selected, randomly combined and mutated to generate the next N architectures (step 406). Over time, this results in architectures which are highly optimized for the given cost functions, which in this case means high accuracy and low implementation/hardware cost. At step 408, a determination is made whether to end. If not, the method 400 proceeds to step 404 and repeats. Otherwise, the method 400 proceeds to step 410, where the training platform outputs the trained neural network.

### Hyperparameter Analysis Based Training

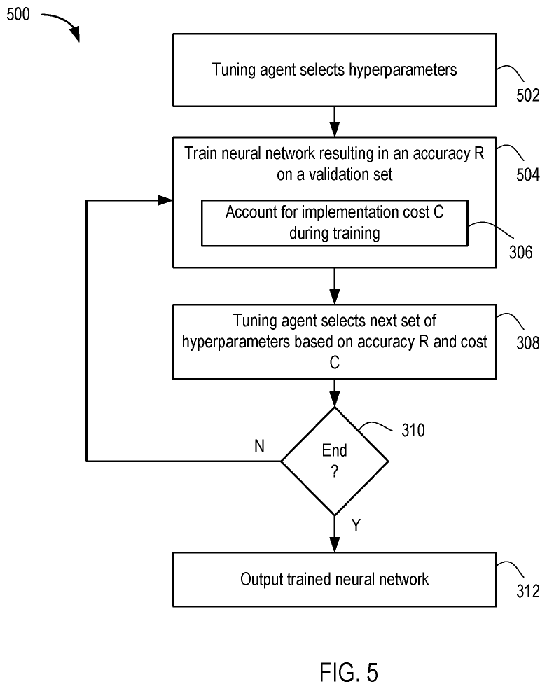

FIG. 5 is a method 500 of training a neural network according to an example. The method 500 begins at step 502, where a tuning agent 105 selects a set of hyperparameters. As noted above, the model-capacity hyperparameters allow definition/description of the architecture of the neural network. The model-capacity hyperparameters define both the topology parameters (e.g., the number of layers, number of channels per layer, etc.) and the related implementation attributes. The tuning agent 105 collects knowledge about the relation between the hyperparameters (both algorithm behavior and model-capacity).

At step 504, the training platform trains the neural network resulting in an accuracy R on a validation set. Since the neural network architecture description includes implementation attributes, the implementation cost C (based on the inference platform) can be measured or estimated/modeled (step 506). At step 508, the tuning agent 105 uses the relation between the hyperparameters and the neural network performance (both accuracy R and the implementation cost C) to make more pareto optimal choices for the next set of hyperparameters. By applying hyperparameter optimization techniques, a good optimum can be achieved in a limited number of optimization steps.

Examples of hyperparameter optimization techniques include grid search, random search, and Bayesian optimization. A grid search involves selecting a set of candidate values for each hyperparameter within a neural network. A grid search is then performed by training a network for each permutation of hyperparameters. The best model is then chosen as the one which performs desirably with respect to our cost functions, described above in the multi-objective optimization section.

A random search is conceptually similar to a grid search, except that a random search picks random values from a specified range for each hyperparameter, rather than selecting them from a grid. This has several benefits including: larger variation in tested hyperparameters, for each hyperparameter, high chance of better performing results than for a grid search, experiments can be interrupted at any point and still be considered a complete set of search data points.

A Bayesian hyperparameter search is a more sophisticated technique which attempts to develop a statistical model which maps the hyperparameter values to our cost function. Usually, this statistical model is a Gaussian Process (GP) which generates functions which closely approximates the observed data. GPs provide a prediction for the chosen cost function in the hyperparameter space, along with the uncertainty of such predictions, this has the following benefits over random search and grid search: 1.) On the next iteration, select a point which minimizes the GP, i.e. the point which is mostly likely to be optimal based on the current model of the hyperparameter space with respect to our desired outcome; and 2.) On the next iteration, select a point with high uncertainty, i.e. a point which will reveal a significant amount of further information about the hyperparameter space.

### Reducing the Architectural Search Space

In the methods above, the size/complexity of the neural architecture search space can be reduced by only making certain aspects of the network variable. For instance, making only the bit width of the feature map elements and the number of channels of the feature maps variable enables training for their optimum setting. Typically, reducing the bit width of the feature map elements results in less accuracy while allowing a more efficient implementation. The reduction in accuracy can be regained by increasing the amount of feature map channels, at the cost of an increased implementation complexity. The feature map element bit width and number of channels can be expressed as part of the neural network architecture description (for the reinforcement learning technique) or as model-capacity hyperparameters (for the hyperparameter analysis). Both techniques for architecture search will explore the (reduced) search space to find a pareto optimal (accuracy versus implementation cost) neural network architecture.

Note that implementations typically come as discrete points in the optimization search space, where an implementation strives to fully exploit the resources of a certain chip/platform. This not only reduces the size of the search space, but also touches another optimization goal of the implementation cost aware network search: maximize the accuracy for that discrete implementation point. This indicates that a listing of the total device resources (for the members of the chip family under consideration) can also become an input to the implementation cost aware architecture search.

Note that, certainly on FPGA architectures, implementation resources, like LUTs, FFs, DSPs, BRAMs/URAMs, etc., typically come in certain ratios for devices within a certain family. These ratios can reduce the number of variables in the multi-objective optimization.

Finally, note that many current neural network topologies do not rely on data-dependent layer executions. This ‘static’ execution of all layers in the neural network simplifies the modeling of the implementation cost of the neural network. If data dependent layer execution is present in the network, a more complex dynamic implementation cost is needed for the neural network architecture search. Alternatively, implementation cost measurements taken while running the topology candidate on the (inference) platform can be used for the neural network architecture search.

### Programmable Device Implementation

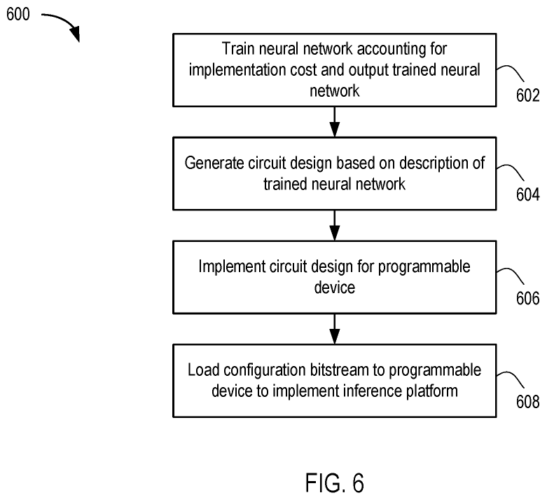

FIG. 6 is a flow diagram depicting a method 600 of implementing an inference platform according to an example. At step 602, the training platform trains a neural network accounting for implementation cost as described in the techniques above. The training platform outputs a trained neural network description. At step 604, a user interacts with circuit design tools to generate a circuit design based on the description of the trained neural network. At step 606, the circuit design tools implement the circuit design for a programmable device, such as an FGPA or an SoC having programmable logic. At step 608, the circuit design tools load the bitstream into a programmable device to implement the inference platform.

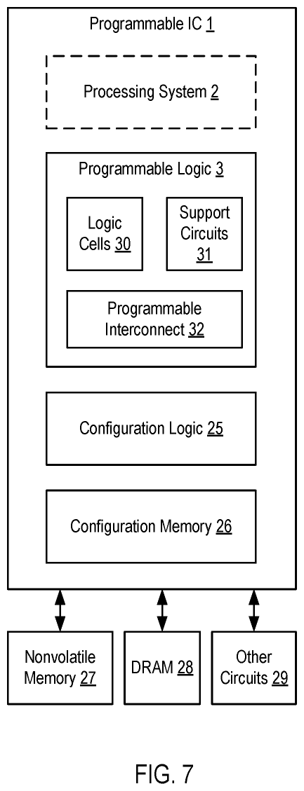

FIG. 7 is a block diagram depicting a programmable IC 1 according to an example that can be used to implement the inference platform and/or training platform. The programmable IC 1 can be used as the IC 220 in FIG. 2. The programmable IC 1 includes programmable logic 3, configuration logic 25, and configuration memory 26. The programmable IC 1 can be coupled to external circuits, such as nonvolatile memory 27, DRAM 28, and other circuits 29. The programmable logic 3 includes logic cells 30, support circuits 31, and programmable interconnect 32. The logic cells 30 include circuits that can be configured to implement general logic functions of a plurality of inputs. The support circuits 31 include dedicated circuits, such as transceivers, input/output blocks, digital signal processors, memories, and the like. The logic cells and the support circuits 31 can be interconnected using the programmable interconnect 32. Information for programming the logic cells 30, for setting parameters of the support circuits 31, and for programming the programmable interconnect 32 is stored in the configuration memory 26 by the configuration logic 25. The configuration logic 25 can obtain the configuration data from the nonvolatile memory 27 or any other source (e.g., the DRAM 28 or from the other circuits 29). In some examples, the programmable IC 1 includes a processing system 2. The processing system 2 can include microprocessor(s), memory, support circuits, IO circuits, and the like.

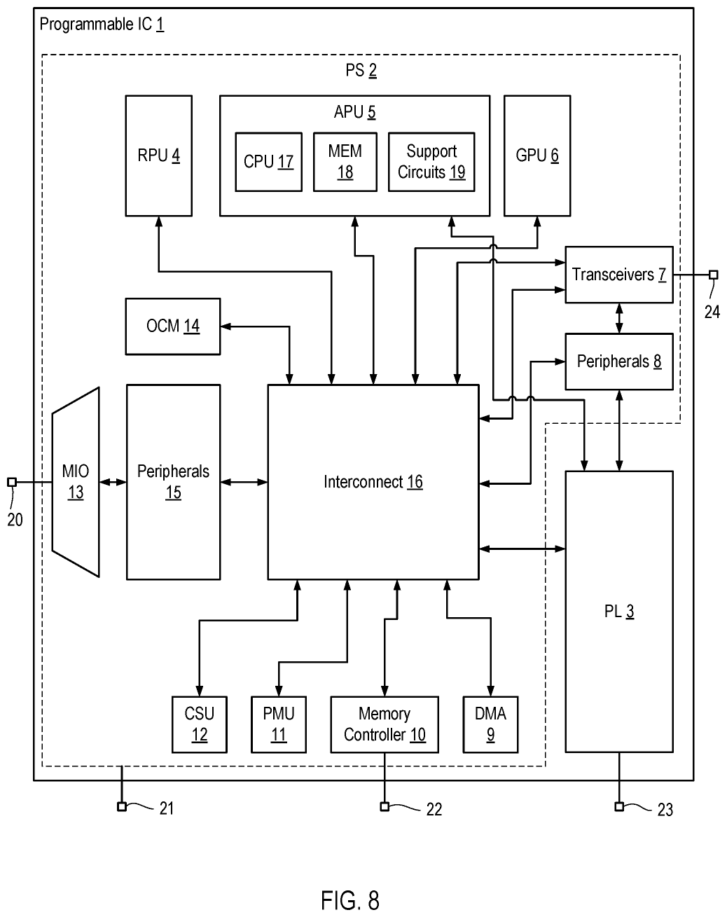

FIG. 8 is a block diagram depicting a System-on-Chip (SoC) implementation of the programmable IC 1 according to an example. In the example, the programmable IC 1 includes the processing system 2 and the programmable logic 3. The processing system 2 includes various processing units, such as a real-time processing unit (RPU) 4, an application processing unit (APU) 5, a graphics processing unit (GPU) 6, a configuration and security unit (CSU) 12, a platform management unit (PMU) 122, and the like. The processing system 2 also includes various support circuits, such as on-chip memory (OCM) 14, transceivers 7, peripherals 8, interconnect 16, DMA circuit 9, memory controller 10, peripherals 15, and multiplexed 10 (MIO) circuit 13. The processing units and the support circuits are interconnected by the interconnect 16. The PL 3 is also coupled to the interconnect 16. The transceivers 7 are coupled to external pins 24. The PL 3 is coupled to external pins 23. The memory controller 10 is coupled to external pins 22. The MIO 13 is coupled to external pins 20. The PS 2 is generally coupled to external pins 21. The APU 5 can include a CPU 17, memory 18, and support circuits 19.

Referring to the PS 2, each of the processing units includes one or more central processing units (CPUs) and associated circuits, such as memories, interrupt controllers, direct memory access (DMA) controllers, memory management units (MMUs), floating point units (FPUs), and the like. The interconnect 16 includes various switches, busses, communication links, and the like configured to interconnect the processing units, as well as interconnect the other components in the PS 2 to the processing units.

The OCM 14 includes one or more RAM modules, which can be distributed throughout the PS 2. For example, the OCM 14 can include battery backed RAM (BBRAM), tightly coupled memory (TCM), and the like. The memory controller 10 can include a DRAM interface for accessing external DRAM. The peripherals 8, 15 can include one or more components that provide an interface to the PS 2. For example, the peripherals 132 can include a graphics processing unit (GPU), a display interface (e.g., DisplayPort, high-definition multimedia interface (HDMI) port, etc.), universal serial bus (USB) ports, Ethernet ports, universal asynchronous transceiver (UART) ports, serial peripheral interface (SPI) ports, general purpose 10 (GPIO) ports, serial advanced technology attachment (SATA) ports, PCIe ports, and the like. The peripherals 15 can be coupled to the MIO 13. The peripherals 8 can be coupled to the transceivers 7. The transceivers 7 can include serializer/deserializer (SERDES) circuits, MGTs, and the like.

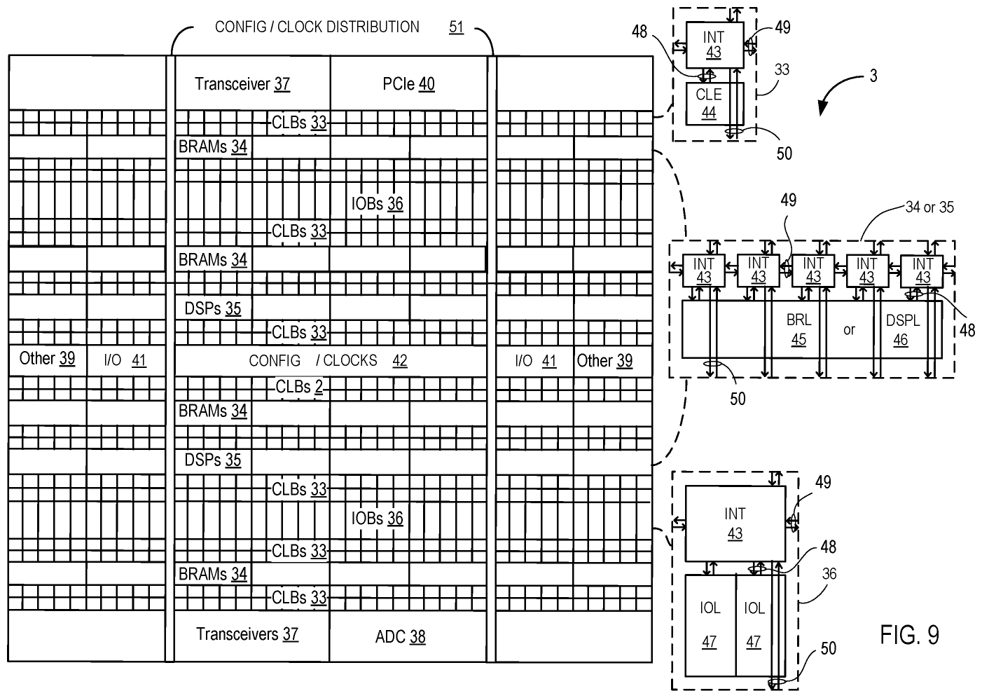

FIG. 9 illustrates a field programmable gate array (FPGA) implementation of the programmable IC 1 that includes a large number of different programmable tiles including transceivers 37, configurable logic blocks (“CLBs”) 33, random access memory blocks (“BRAMs”) 34, input/output blocks (“IOBs”) 36, configuration and clocking logic (“CONFIG/CLOCKS”) 42, digital signal processing blocks (“DSPs”) 35, specialized input/output blocks (“I/O”) 41 (e.g., configuration ports and clock ports), and other programmable logic 39 such as digital clock managers, analog-to-digital converters, system monitoring logic, and so forth. The FPGA can also include PCIe interfaces 40, analog-to-digital converters (ADC) 38, and the like.

In some FPGAs, each programmable tile can include at least one programmable interconnect element (“INT”) 43 having connections to input and output terminals 48 of a programmable logic element within the same tile, as shown by examples included at the top of FIG. 9. Each programmable interconnect element 43 can also include connections to interconnect segments 49 of adjacent programmable interconnect element(s) in the same tile or other tile(s). Each programmable interconnect element 43 can also include connections to interconnect segments 50 of general routing resources between logic blocks (not shown). The general routing resources can include routing channels between logic blocks (not shown) comprising tracks of interconnect segments (e.g., interconnect segments 50) and switch blocks (not shown) for connecting interconnect segments. The interconnect segments of the general routing resources (e.g., interconnect segments 50) can span one or more logic blocks. The programmable interconnect elements 43 taken together with the general routing resources implement a programmable interconnect structure (“programmable interconnect”) for the illustrated FPGA.

In an example implementation, a CLB 33 can include a configurable logic element (“CLE”) 44 that can be programmed to implement user logic plus a single programmable interconnect element (“INT”) 43. A BRAM 34 can include a BRAM logic element (“BRL”) 45 in addition to one or more programmable interconnect elements. Typically, the number of interconnect elements included in a tile depends on the height of the tile. In the pictured example, a BRAM tile has the same height as five CLBs, but other numbers (e.g., four) can also be used. A DSP tile 35 can include a DSP logic element (“DSPL”) 46 in addition to an appropriate number of programmable interconnect elements. An 10B 36 can include, for example, two instances of an input/output logic element (“IOL”) 47 in addition to one instance of the programmable interconnect element 43. As will be clear to those of skill in the art, the actual I/O pads connected, for example, to the I/O logic element 47 typically are not confined to the area of the input/output logic element 47.

In the pictured example, a horizontal area near the center of the die (shown in FIG. 9) is used for configuration, clock, and other control logic. Vertical columns 51 extending from this horizontal area or column are used to distribute the clocks and configuration signals across the breadth of the FPGA.

Some FPGAs utilizing the architecture illustrated in FIG. 9 include additional logic blocks that disrupt the regular columnar structure making up a large part of the FPGA. The additional logic blocks can be programmable blocks and/or dedicated logic.

Note that FIG. 9 is intended to illustrate only an exemplary FPGA architecture. For example, the numbers of logic blocks in a row, the relative width of the rows, the number and order of rows, the types of logic blocks included in the rows, the relative sizes of the logic blocks, and the interconnect/logic implementations included at the top of FIG. 9 are purely exemplary. For example, in an actual FPGA more than one adjacent row of CLBs is typically included wherever the CLBs appear, to facilitate the efficient implementation of user logic, but the number of adjacent CLB rows varies with the overall size of the FPGA.

The various examples described herein may employ various computer-implemented operations involving data stored in computer systems. For example, these operations may require physical manipulation of physical quantities—usually, though not necessarily, these quantities may take the form of electrical or magnetic signals, where they or representations of them are capable of being stored, transferred, combined, compared, or otherwise manipulated. Further, such manipulations are often referred to in terms, such as producing, identifying, determining, or comparing. Any operations described herein that form part of one or more examples techniques described herein may be useful machine operations. In addition, one or more example techniques also relate to a device or an apparatus for performing these operations. The apparatus may be specially constructed for specific required purposes, or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations. The various examples described herein may be practiced with other computing system configurations including hand-held devices, microprocessor systems, microprocessor-based or programmable consumer electronics, minicomputers, mainframe computers, and the like.

One or more example techniques described herein may be implemented as one or more computer programs or as one or more computer program modules embodied in one or more computer readable media. The term computer readable medium refers to any data storage device that can store data which can thereafter be input to a computer system—computer readable media may be based on any existing or subsequently developed technology for embodying computer programs in a manner that enables them to be read by a computer. Examples of a computer readable medium include a hard drive, network attached storage (NAS), read-only memory, random-access memory (e.g., a flash memory device), a CD (Compact Discs)—CD-ROM, a CD-R, or a CD-RW, a DVD (Digital Versatile Disc), a magnetic tape, and other optical and non-optical data storage devices. The computer readable medium can also be distributed over a network coupled computer system so that the computer readable code is stored and executed in a distributed fashion.

While the foregoing is directed to specific examples, other and further examples may be devised without departing from the basic scope thereof, and the scope thereof is determined by the claims that follow.

What is claimed is:

1. A method of implementing a neural network, comprising:
selecting a first neural network architecture from a search space;
training the neural network having the first neural network architecture to obtain an accuracy and an implementation cost, the implementation cost based on a programmable device of an inference platform;
selecting a second neural network architecture from the search space based on the accuracy and the implementation cost; and
outputting weights and hyperparameters for the neural network having the second neural network architecture.

2. The method of claim 1, wherein the step of selecting the first neural network architecture is performed by a reinforcement agent 103, wherein the reinforcement agent 103 selects the first neural network architecture from the search space with a probability P, and wherein the reinforcement agent 103 adjusts the probability P based on a function of the accuracy and the implementation cost.

3. The method of claim 1, wherein the reinforcement agent 103 is a recurrent neural network (RNN).

4. The method of claim 1, wherein the first neural network architecture is one of a plurality of neural network architectures, wherein the step of training includes evaluating the plurality of neural network architectures using a fitness function.

5. The method of claim 1, wherein the step of selecting the first neural network architecture is performed by a tuning agent 105, and wherein the tuning agent 105 selects hyperparameters for the second neural network architecture based on a function of the accuracy and the implementation cost.

6. The method of claim 5, wherein the tuning agent 105 selects the hyperparameters using a grid search, random search, or Bayesian search.

7. The method of claim 1, further comprising:
generating a circuit design based on the weights and the hyperparameters of the neural network; and
implementing the circuit design for the programmable logic device.

8. A non-transitory computer readable medium comprising instructions, which when executed in a computer system, causes the computer system to carry out a method of implementing a neural network, comprising:
selecting a first neural network architecture from a search space;
training the neural network having the first neural network architecture to obtain an accuracy and an implementation cost, the implementation cost based on a programmable device of an inference platform;
selecting a second neural network architecture from the search space based on the accuracy and the implementation cost; and
outputting weights and hyperparameters for the neural network having the second neural network architecture.

9. The non-transitory computer readable medium of claim 8, wherein the step of selecting the first neural network architecture is performed by a reinforcement agent 103, wherein the reinforcement agent 103 selects the first neural network architecture from the search space with a probability P, and wherein the reinforcement agent 103 adjusts the probability P based on a function of the accuracy and the implementation cost.

10. The non-transitory computer readable medium of claim 8, wherein the reinforcement agent 103 is a recurrent neural network (RNN).

11. The non-transitory computer readable medium of claim 8, wherein the first neural network architecture is one of a plurality of neural network architectures, wherein the step of training includes evaluating the plurality of neural network architectures using a fitness function.

12. The non-transitory computer readable medium of claim 8, wherein the step of selecting the first neural network architecture is performed by a tuning agent 105, and wherein the tuning agent 105 selects hyperparameters for the second neural network architecture based on a function of the accuracy and the implementation cost.

13. The non-transitory computer readable medium of claim 12, wherein the tuning agent 105 selects the hyperparameters using a grid search, random search, or Bayesian search.

14. The non-transitory computer readable medium of claim 8, further comprising:
generating a circuit design based on the weights and the hyperparameters of the neural network; and
implementing the circuit design for the programmable logic device.

15. A computer system, comprising:
a memory having program code stored therein; and
a processor, configured to execute the program code, to implement a neural network by:
selecting a first neural network architecture from a search space;
training the neural network having the first neural network architecture to obtain an accuracy and an implementation cost, the implementation cost based on a programmable device of an inference platform;
selecting a second neural network architecture from the search space based on the accuracy and the implementation cost; and
outputting weights and hyperparameters for the neural network having the second neural network architecture.

16. The computer system of claim 15, wherein the processor is configured to execute the code to select the first neural network architecture using a reinforcement agent 103, wherein the reinforcement agent 103 selects the first neural network architecture from the search space with a probability P, and wherein the reinforcement agent 103 adjusts the probability P based on a function of the accuracy and the implementation cost.

17. The computer system of claim 15, wherein the reinforcement agent 103 is a recurrent neural network (RNN).

18. The computer system of claim 15, wherein the first neural network architecture is one of a plurality of neural network architectures, wherein the processor executes the code to perform the training by evaluating the plurality of neural network architectures using a fitness function.

19. The computer system of claim 15, wherein the processor executes the code to select the first neural network architecture using a tuning agent 105, and wherein the tuning agent 105 selects hyperparameters for the second neural network architecture based on a function of the accuracy and the implementation cost.

20. The computer system of claim 19, wherein the tuning agent 105 selects the hyperparameters using a grid search, random search, or Bayesian search.
